(19) United States
(12) Patent Application Publication
(10) Pub. No.: US 2013/0064344 A1
(43) Pub. Date: Mar. 14, 2013

(54) Vertical Imaging of an Upright Patient

(71) Applicant: Mark Carol, Burlingame, CA (US)

(72) Inventor: Mark Carol, Burlingame, CA (US)

(21) Appl. No.: 13/588,009

(22) Filed: Aug. 17, 2012

Related U.S. Application Data
(60) Provisional application No. 61/526,101, filed on Aug. 22, 2011.

(51) Int. Cl. A61B 6/03

(52) U.S. Cl. 378/10

(57) ABSTRACT

A CT scanning system for patients is provided comprising a nonrotating x-ray source and detector array bracketing a turntable on which stands or sits the patient that is being imaged. As the patient rotates between the x-ray source and the detector array that move along the vertical axis in tandem for each angular position of the patient, imaging data is collected of the patient from multiple angles at multiple levels that are reconstructed to form computed tomographic images that cover the entire region of interest.

### CROSS-REFERENCE TO RELATED APPLICATIONS

This application is entitled to the benefit of Provisional Patent Application 61/526,101 filed Aug. 22, 2011.

### BACKGROUND

1. Field of Invention

This invention relates to imaging patients with computed tomography, specifically to imaging patients in the sitting or standing position, rotating the patient in front of imaging components that themselves do not rotate, with or without a therapy component.

In the early days of tomographic imaging, equipment was very time consuming to use, requiring up to an hour or more to image a patient. Medical diagnostic tomographic apparatuses originated from the concept of a stationary detector disclosed in the original Anger patents, such as U.S. Pat. No. 3,432,660 related to tomographic imaging using radioactive materials and radiation sensitive scintillation crystals. In these early days of gamma camera imaging an approach was used whereby the patient sat in front of a camera and the patient was rotated in order to generate multiple views. Such a device was described by Muehllehner and Wetzel and was also investigated by Budinger, Gullberg and Huesman in late 1960s and early 1970s and taught in U.S. Pat. No. 5,036,530. The approaches and teachings were limited to radioactive sources and were not referenced to be used with x-rays, nor was it applicable to the entire anatomy, being restricted only to the head and brain.

In addition, advanced imaging technology in medicine also was original meant for use on critically ill patients and was rarely used on outpatients. Therefore, the recumbent position was more appropriate in order to keep patients comfortable and for use on those who were comatose or could only be cared for in the recumbent position.

As imaging technology has advanced over the years, it is now at the point where CT images using x-rays can be generated in seconds. CT using x-rays has become more ubiquitous and is considered standard of care for imaging in almost every medical ailment. In the current practice of computed tomographic imaging, a kilovoltage x-ray source is paired with a means of radiation detection to acquire the data used to create images. Typically, an array of discrete detector elements will be used to detect the radiation that passes through the object being imaged. This array can have anywhere from one to 128 or more rows of detectors that can be spaced a variable distance apart. In order to acquire data over an extended length of patient anatomy, the detector array and the x-ray source are moved relative to the patient typically by moving the patient through the gantry containing the x-ray source and detector array. With modern high speed CT scanners capable of generating hundreds of very thin slice at a time, scans covering long distance of patient anatomy can be achieved in a very short time.

This approach is less than ideal because a recumbent patient requires significant floor space and also because it is less comfortable and convenient for a patient to lie down than to sit or stand. The vast majority of patients who receive an x-ray CT are imaged electively, walking in and out of the imaging facility under their power. Immobilization devices exist, such as U.S. Pat. Nos. 4,400,820, 4,481,657, 4,779,858, designed to hold patients still when lying down, and U.S. Pat. No. 5,036,530, that can hold patients still for even prolonged periods of time regardless of their position. Yet these imaging studies are still performed on recumbent patients in a room that is large enough to accommodate a recumbent patient.

Therefore it has become desirable to provide a means of performing a CT using a geometry that is more cognizant of the universality and outpatient applications of tomographic imaging, that takes advantages of the improvements that have been made in image acquisition and data processing, and that can be deployed in a minimum amount of space. This geometry involves the upright or vertical (sitting and standing) positions.

2. Background Description of Prior Art

U.S. Pat No. 4,316,091 teaches a CT scanner in which components are mounted in an apertured housing that can be deployed generally vertically, for the examination of recumbent patients, or generally horizontally, for the examination of patients seated, or standing. U.S. Pat. No. 5,042,487 teaches the use of a vertically-oriented and tiltable chair in which a patient can be imaged using a horizontally deployed CT gantry. U.S. Pat. No. 5,574,763 describes a CT having a patient support which permits a scan to be conducted of a patient with the patient in a substantially upright, but slightly tilted, position, and a control unit to maintain the gantry containing the x-ray source and detector at the same angle relative to said patient support for all positions of the patient support during a scan. U.S. Pat. No. 7,003,070 describes an upright CT scanner where an X-ray source and a two-dimensional digital X-ray detector are mounted on a rotating platform with avertical axis of rotation so as to rotate the imaging source/detector array about a patient. U.S. Pat. No. 7,224,764 describes an upright scanner where the source and detector rotates about a generally upright axis and is used to scan the spine and extremities, and U.S. Pat. No. 7,460,637 describes a similar device teaching the imaging of a patient's head and face with the patient in the sitting position and the x-ray source and detector rotating about the patient on a gantry. U.S. Pat. No. 7,224,764 describes a CT scanner that is particularly useful for scanning the spine and extremities, such as knees, and ankles, especially while the patient is in an upright position. The CT scanner generally includes a source and detector that are rotatable about a generally upright axis. The source and detector are also moved along the upright axis during rotation to perform a helical scan. Kamada et all published in 1999 on the use of a horizontal helical CT system for 3-D treatment planning and for positioning verification of patients in the seated position (Radiotherapy and Oncology 50 (1999) 235-237; A horizontal CT system dedicated to heavy-ion beam treatment Tadashi Kamadaa,*, Hirohiko Tsujiia, Jun-Etsu Mizoea, Yoshisuke Matsuokaa, Hiroshi Tsujia, Yasuhiro Osakaa, Shinichi Minoharab, Nobuyuki Miyaharab, Masahiro Endob, Tatsuaki Kanai). Shah et al. described the use of a horizontally deployed CT gantry to image a patient in the vertical position for the purposes of radiation therapy treatment planning (Upright 3D treatment planning using a vertical CT. Shah A P, Strauss J B, Kirk M C, Chen S S, Kroc T K, Zusag T W. Med Dosim. 2009 Spring;34(1):82-6). All of these approaches, although able to generate fan-beam diagnostic quality images, require a complicated gantry arrangement that can rotate about a patient, making it difficult to deploy this kind of technology in the therapy suite where the imaging device can interfere with the therapy beam.

U.S. Pat. No. 7,796,730 discloses an approach designed to solve this problem, teaching the use of a CT scanner with respect to a sitting patient for radiation therapy whereby the scanner can be removeably positioned to image the patient. When in place for imaging it obscures the treatment beam, when removed it allows the patient to be imaged. Although the patient can rotate in front of the imaging beam to generate an image, the imaging device completely surrounds the patient and must be removed, or must provide a window through it, for the patient to be treated.

So called rotating patient CT scanners are disclosed in U.S. Pat. Nos. 4,473,822; 4,961,208; 5,036,530. Patient is positioned in an upright position between x-ray source and bank of detectors. For a given vertical position of the x-ray source and the detectors, the patient is rotated 360 degrees to acquire multiple images from different angular positions of the patient. This provides the data to generate anywhere from one to many images slices through the patient depending on the number of rows of detectors employed. Once a complete rotation of the patient is finished, the x-ray source and detectors are moved together, relative to the patient, along a vertical translation axis to generate a new set of horizontal scan planes by rotating the patient again. This process is repeated until the complete area of interest is covered. This approach generates high diagnostic quality images due to the use of fan-beam technology but requires that the patient be rotated multiple times in order to achieve that quality, something that can cause undue discomfort to the patient and increase the time it takes to generate a study.

U.S. Pat. No. 6,470,068 teaches the use of the vertical position with a patient mounted on a turntable, the patient held in place using an airbag system, and a conventional CT source/detector array configuration. The system teaches the use of a cone beam technology in order to generate images quickly and cheaply with the need to rotate the patient only once. However, cone beam technology does not generate images equal in diagnostic quality to those generated by fan beam technology.

In the industrial world CT is particularly useful for nondestructively revealing the internal structure of industrial products. X-ray inspection systems for large component objects, such as those systems disclosed in U.S. Pat. Nos. 3,766,387; 4,422,177; 4,600,998; 5,119,408; and 7,760,852 use a system where an x-ray source and a beam detector system straddle a turntable on which is placed an industrial object for imaging that is rotated in front of the x-ray source and beam detector for the purposes of generating a CT dataset. For instance, U.S. Pat. No. 5,023,895 teaches an industrial CT system is provided for three-dimensional imaging that includes a three dimensional cone beam of hard radiation fixed with respect to a two dimensional scintillation detector array. The object is positioned on a turntable interposed between the radiation source and detector array. Data from two-dimensional views are stored as the object is rotated on the turntable about a fixed axis. The data is sufficient upon completion of one revolution to construct a transparent three-dimensional image of the object. A positioning encoding arrangement adjusts for variations in the object's mass density to optimize scan-compute times while enhancing image resolution. However, none of these teach the use of such a geometry for the purposes of medical imaging nor do they discuss how to generate fan beam images.

U.S. Pat. Nos. 6,649,914; 5,859,893; 5,835,561; 5,751,785; 5,729,584; 5,651,047; 5,644,612; 5,610,967 all relate to the generation of x-ray images using an inverse geometry. This geometry is characterized by a large-area scanning x-ray source and a small-area detector. The scanning x-ray source projects a rapid sequence of narrow x-ray beams through the patient and onto a small-area, high efficiency detector. The system acquires a series of overlapping small-field-of-view images that are combined electronically to generate the full image. Although this technology has been described as being used to generate CT images, it does so by rotating the x-ray source and the detector about the patient. None of the patents teach the approach of rotating the patient in front an x-ray source and the detector array instead of rotating the x-ray source and the detector array.

Therefore, although there is teaching related to the standing or sitting position for the purposes of CT fan beam imaging where the imaging components rotate about the patient, and although there is teaching related to fan-beam imaging with a rotating patient where multiple rotations of the patient are required to complete a scan, and although there is teaching related to the requirement for only a single rotation of a patient in a cone-beam imaging system, and although there is teaching related to the use of system whereby an industrial object rotates between the components of an imaging system, there is no prior art teaching the use of a nonrotating detector/array assembly imaging with a rotating patient in either the horizontal or vertical positions where only a single rotation of a patient is required to acquire fan beam images.

Thus there is a need for and novel design in a CT scanning system that provides the ability to image a patient in the standing or upright position using nonrotating imaging components whereby high quality fan beam images are acquired with a single rotation of a patient, thereby allowing a CT system to be deployed in a minimum amount of space and with a minimum amount of cost and a minimum of inconvenience to the patient. This is especially the case for the purposes of imaging a patient who is undergoing therapeutic radiation therapy.

### SUMMARY

A CT scanning system for patients is provided comprising a nonrotating x-ray source and detector array bracketing a turntable on which stands or sits the patient that is being imaged. As the patient rotates between the x-ray source and the detector array that move along the vertical axis in tandem for each angular position of the patient, imaging data is collected of the patient from multiple angles at multiple levels that are reconstructed to form computed tomographic images that cover the entire region of interest.

In a preferred embodiment the system consists of a turntable interposed between two imaging components that can support a patient sitting or standing. The imaging components consists of a detector array characterized by an array of discrete detector elements typical to modern day CT scanners, and an x-ray source, the two components able to be moved in tandem vertically up and down for each angular position of the rotating patient so as to generate overlapping small-fields-of-view whose data can be combined electronically to generate a complete data set from each angular data collection position.

In further embodiment the imaging components consist of an inverse geometry imaging system characterized by a large-area scanning x-ray source and a small-area detector, the scanning x-ray source projecting a rapid sequence of narrow x-ray beams through the patient at each angular position of the rotating patient onto a small-area, high efficiency detector so as to generate overlapping small-fields-of-view whose data can be combined electronically to generate a complete data set from each angular data collection position.

In one aspect, a method is provided for generating a CT image of a patient. The method comprises the steps of locating a patient upright or sitting on a turntable that is positioned between an x-ray source and an x-ray detector of an imaging system, rotating the patient such that the imaging beam generated by the x-ray source passes through different angular positions of the patient before reaching the detector, translating the detector array and x-ray source along a vertical axis to generate multiple images of the patient displaced in a vertical direction for each angular position of the patient, the data from all positions of the patient and all vertical positions of the x-ray source and detector array being combined to generate the CT images in a single rotation of the patient.

A further method comprising the steps of locating a patient upright or sitting on a turntable that is positioned between the two imaging components of an inverse geometry imaging system and rotating the patient while collecting data at each angular position of the patient that are combined to generate the CT images, thereby generating the equivalent of fan-beam imaging with only a single rotation of the patient.

### DRAWINGS

FIG. 1 depicts the various components of a preferred embodiment of the invention using a conventional imaging geometry.

FIG. 2 depicts the various components of an embodiment of the invention using an inverse imaging geometry.

FIG. 3 depicts the various components of the integrated system of vertically moving detector and x-ray source tandem and the patient support platform with turntable and x-y table

FIG. 4 depicts the relationship between the imaging tandem and a therapy unit

FIG. 5 depicts the vertical movement of the imaging components used to cover the entire region of interest

FIG. 6 depicts a preferred embodiment for moving the imaging tandem components

FIG. 7 depicts an alternative embodiment for moving the imaging tandem components

FIG. 8 depicts the vertical and x-y movement of the patient support platform

FIG. 9a through FIG. 9d depict the relationship between angular position of the patient support platform as it rotates between the source and detector tandem and the vertical position of the source and detector tandem.

### DETAILED DESCRIPTION OF VARIOUS EMBODIMENTS

Various embodiments of the present invention are described hereinafter with reference to the figures. It should be noted that the figures are not drawn to scale and elements of similar structures or functions are represented by like reference numerals throughout the figures. It should also be noted that the figures are not intended to facilitate the description of specific embodiments of the invention. They are not intended as an exhaustive description of the invention or as a limitation on the scope of the invention. In addition an aspect described in conjunction with a particular embodiment of the present invention is not necessarily limited to that embodiment and can be practiced in any other embodiments of the present invention. It will be appreciated that while various embodiments of the invention are described in connection with computed tomographic imaging in medicine, the claimed invention has application in other industries and to imaging technologies other than computed tomography.

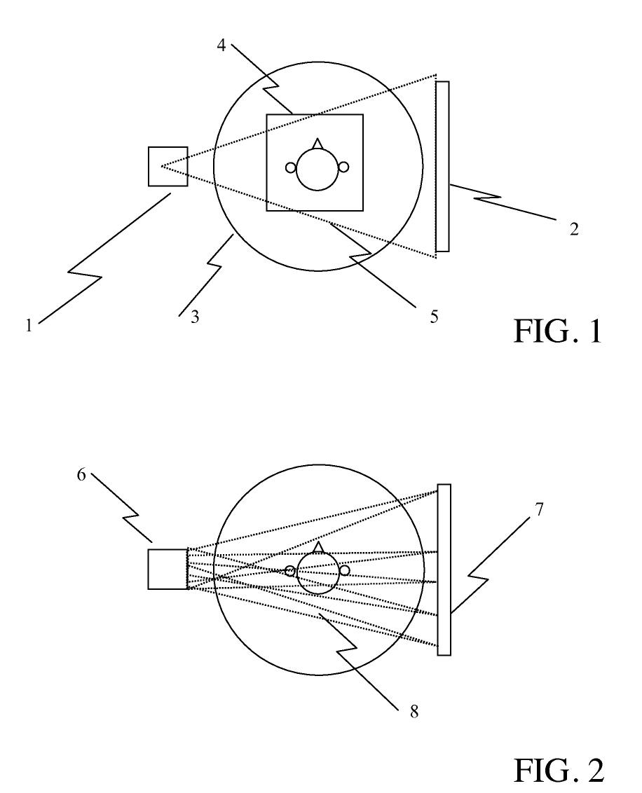

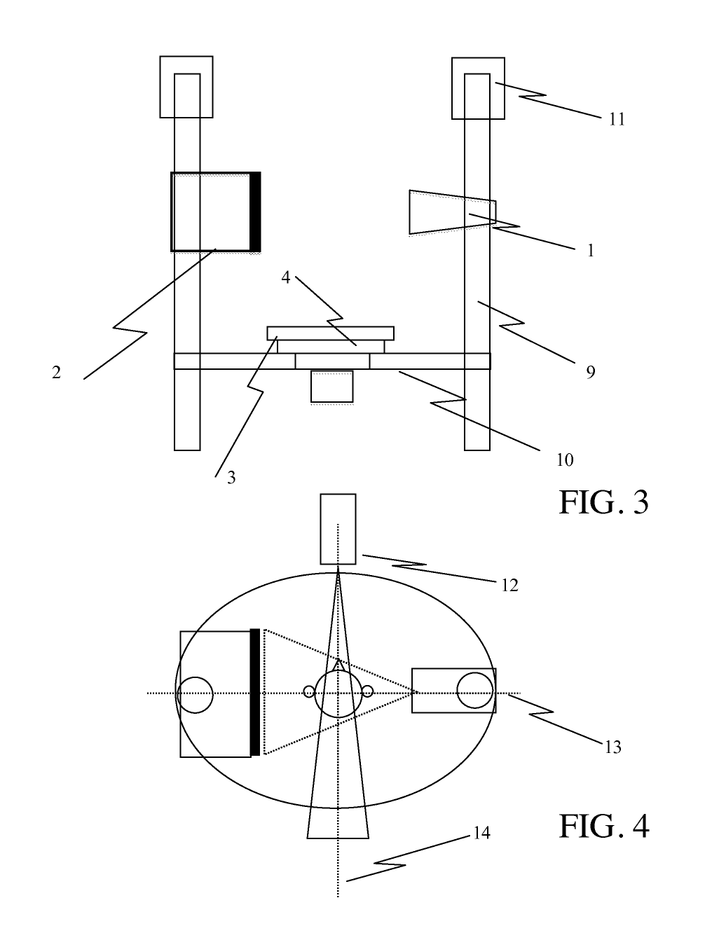

In a preferred embodiment of the present invention as depicted in FIG. 1, a patient support platform (4) is mounted on a turntable (3) that is interposed between an x-ray source (1) and an array of detectors (2) positioned such that the line connecting the two is orthogonal to the axis of rotation of the turntable. The detector array can consist of any number of rows of detectors as is standard in the industry. The x-ray source is any diagnostic quality x-ray source as is used typical in the industry. The x-ray source and the detector array are mounted on a mechanism that allows them to translate in tandem along a vertically axis: An example of such a mechanism is depicted in FIG. 3 and consists of two vertical posts (9) on which the imaging components can travel through the use of a guide screw and motor (11). Any other such mechanism well known to those skilled in the art can be used as well. The turntable is mounted on a platform, such as an x-y table (4), that allows it to be translated with at least two degrees of freedom consisting of in/out and right/left relative to the x-ray source, and preferably three, adding translation along the axis of rotation. This vertical degree of freedom can be implemented using the same vertical posts on which the imaging components move, as depicted in FIG. 8, through the use of a support plate (10) that connects with the vertical posts or any other means well know to those skilled in the art. The turntable can rotate continuously or can be indexed in angular increments.

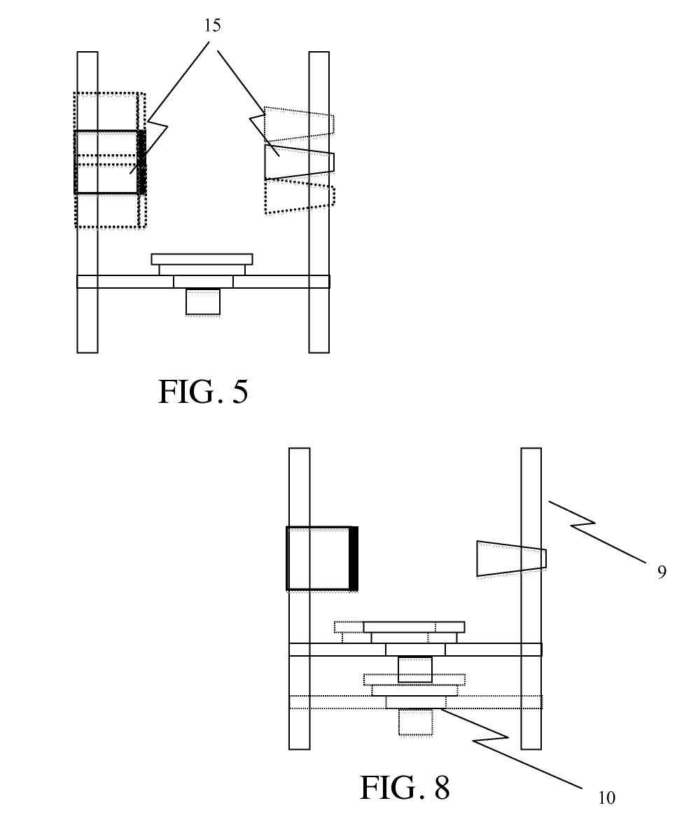

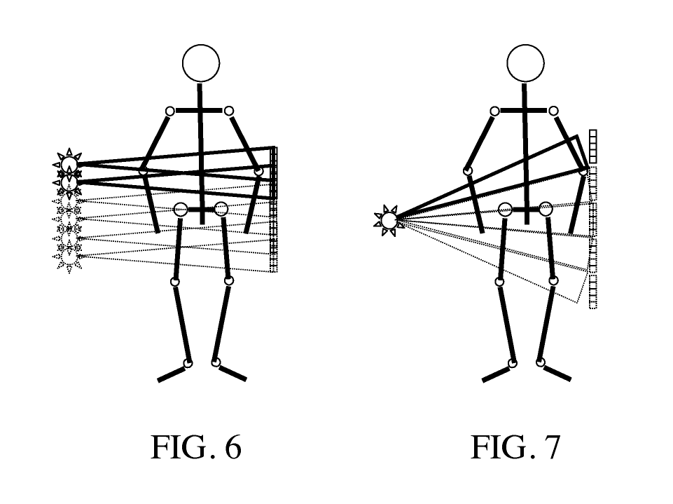

If the imaging system is to be used in conjunction with a therapy system (12) such as ionizing radiation as in FIG. 4, the imaging components are mounted such that the plane defining the x-ray source and detector (13) is orthogonal to the therapy beam path (14). The system includes a control component that coordinates movement of the turntable with that of the imaging tandem. The movement of the imaging tandem along an axis orthogonal to the axis of rotation of the turntable, as in FIG. 5, can be associated with the angular position of the turntable such that for each angular position of the turntable the imaging tandem consisting of an x-ray source and a detector array (15) can be moved up or down, as in FIG. 6, to cover the complete region to be imaged, the amount of movement required being related to the number of rows in the detector array and the length of the region to be covered. Various means can be provided for immobilizing a patient on the patient support platform in either the sitting or standing position. Other means can be provided for positioning the imaging platform correctly relative to the imaging tandem so that the correct and desired region of anatomy can be imaged.

In a variant of the preferred embodiment as depicted in FIG. 7, the x-ray source of the imaging components can angle along the vertical plane rather than translate along the vertical axis. The detector array translates along the vertical axis in tandem with the angulation of the x-ray source such that the imaging beam always impinges on the detector array.

In an additional embodiment, as depicted in FIG. 2, an inverse geometry imaging system is used as described in U.S. Pat. Nos. 6,649,914; 5,859,893; 5,835,561; 5,751,785; 5,729,584; 5,651,047; 5,644,612; 5,610,967, 5,267,296, and 3,949,229. A patient support platform is mounted on a turntable that is interposed between a large-area scanning x-ray source (7) and a small-area detector (6) such that the line connecting the two is orthogonal to the axis of rotation of the turntable. The scanning x-ray source projects a rapid sequence of narrow x-ray beams (8) through the patient and onto the small-area, high efficiency detector so as to cover a variable region of interest whose dimensions are determined by the size of the components. The scanning x-ray source and the small-area detector can be moved in tandem along the vertical axis in order to be positioned properly to cover the region of interest. The turntable is mounted on a platform that allows it to be translated with at least two degrees of freedom consisting of in/out and right/left relative to the x-ray source, and preferably three, adding translation along the axis of rotation. The turntable can rotate continuously or can be indexed in angular increments. If the imaging system is to be used in conjunction with a therapy system such as ionizing radiation, the imaging components are mounted such that the line connecting the x-ray source and detector is orthogonal to the therapy beam path. The system includes a control component that positions the imaging component and turntable position such that the imaging beams will pass through the region of interest. In those cases where the desired field of view is greater than the maximum field of view of the imaging device, the x-ray source and detector tandem can be translated along the vertical axis and the horizontal axis in order to cover the desired field of view. This translation is coordinated with the angular position of the turntable so that for each angular position of the turntable the entire field of view is covered by the imaging beams. Various means can be provided for immobilizing a patient on the patient support platform in either the sitting or standing position. Other means can be provided for positioning the imaging platform correctly relative to the imaging tandem so that the correct and desired region of anatomy can be imaged.

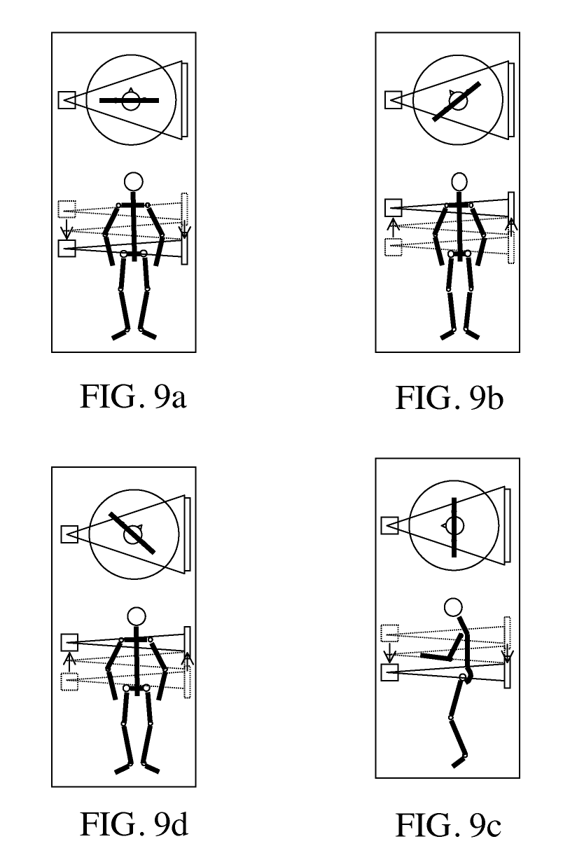

In operation using the preferred embodiment as shown in FIG. 9, a patient is positioned on the patient support platform in either the sitting or standing or semi-standing position and may be immobilized in that position. A localizing x-ray is taken by moving the imaging tandem with the x-ray source turned on in a vertical direction so as to cover the entire general region of interest with the imaging beam without rotating the turntable. The acquired data is processed to generate a 2-D image of the patient so that he region of interest for 3-D imaging can be identified. The position of the turntable is adjusted so that the region of interest is centered relative to the position of the imaging tandem. The imaging tandem is then moved along the vertical axis so that it is in-line with the inferior margin of the region of interest.

With the imaging beam turned on, the 3-D data acquisition process begins by moving the imaging tandem along the vertical axis in fixed increments until the complete length of the region of interest is covered (FIG. 9a). The size of the fixed increment is related to the number of rows of detectors in the detector array and the desired resolution, in terms of slice thickness, of the imaging study being performed. The data generated during this process is tagged with the angular position of the turntable and the vertical position of the imaging tandem. The turntable is then rotated by a set amount and the imaging tandem is translated in a similar fashion along the vertical axis back to its starting point (FIG. 9b) with the data being tagged as before. This angular indexing of the turntable with vertical indexing of the imaging tandem for each angular position of the turntable continues (FIG. 9c-d) until a full rotation of the turntable has been achieved. The patient can then be removed from the turntable if undergoing only an imaging study. If the patient is to be treated using data resulting from the imaging procedure, such as by a therapy beam of radiation that is to be targeted based on positional data on the patient or the target to be treated derived from the imaging study, the patient would remain on the turntable, immobilized in the same position as imaged, so that treatment can proceed using the targeting data generated by the imaging procedure.

The data acquired during the imaging procedure is then parsed according to the turntable's angular position and the imaging tandem's vertical position. All of the images generated for a given vertical position of the imaging tandem when combined will provide the data required to generate, using techniques and algorithms known to those skilled in the art, a set of imaging slices through the patient corresponding to that vertical position of the imaging tandem. If a detector array with multiple rows of detectors is provided, then multiple image slices will be generated for each vertical position of the imaging tandem.

In an alternate version of the preferred embodiment, the operation is the same except that instead of translating the x-ray source in tandem with the detector array, the x-ray source is angled along the vertical axis so as to track the position of the detector array as it translates along the vertical axis.

In another embodiment employing inverse geometry imaging, indexing of the imaging tandem for each angular position of the turntable occurs only if the field of view of the imaging system is not sufficient to cover the region of interest to be imaged. If such a case exists, the imaging tandem can be indexed for each angular position of the turntable so as to cover the entire region of interest with the imaging beam for ach angular position of the turntable. This indexing can occur along the vertical, the horizontal, or both the vertical and horizontal axis as required to achieve complete coverage.

In any of the embodiments, rather the using a fixed increment for both the turntable rotation and the vertical translation of the imaging tandem, a continuous rotation of the turntable and a continuous translation of the imaging tandem can be employed. Although the algorithms for image reconstruction are different than in the fixed increment approach, such algorithms are well know to those skilled in the art.

It will be appreciated that while various embodiments are described for this invention, they are not intended as an exhaustive description of the invention or as a limitation on the scope of the invention. For instance, multiple x-ray sources or multiple detector arrays can be used to limit the amount of indexing of the imaging tandem required to achieve full coverage of the region of interest.

I claim:

1. An apparatus for performing imaging of patient, said apparatus comprising:
a. A x-ray source and detector
b. A turntable bracketed by the x-ray source and detector
c. Means for supporting on the turntable a patient in a generally sitting or standing position
d. Means for positioning of the x-ray source and detector
e. Means for aggregating the generated imaging data
whereby the x-ray source and detector are located so as to bracket the region of interest and generate imaging data on the patient as the turntable makes a single rotation in front of the x-ray source, said generated being processed to produce images that cover the region of interest.

2. The apparatus of claim 1 wherein said detector consists of at least a one dimension array of discrete detector elements

3. The apparatus of claim 1 wherein said x-ray source consists of a large-area scanning x-ray source and said detector consists of a small area-detector

4. The apparatus of claim 1 wherein said means for positioning provides tandem vertical and horizontal movement of said source and said detector so that the x-ray beam intersects cumulatively the entire region of interest for each angular position of the turntable.

5. The apparatus of claim 1 wherein said means for positioning provides for angular positioning of said source and vertical and horizontal movement of said detector so that the detector intercepts the imaging beam after it passes through the patient whereby the x-ray encompasses cumulatively the entire region of interest for each angular position of the patient.

6. The apparatus of claim 1 wherein said means for positioning provides for angular positioning of said detector and vertical and horizontal movement of said source so that the detector intercepts the imaging beam after it passes through the patient whereby the x-ray encompasses cumulatively the entire region of interest for each angular position of the patient.

7. The apparatus of claim 1 when used in conjunction with a radiation therapy system whereby the plane defining the x-ray beam and detector is orthogonal to the therapy beam direction.

8. A method for performing x-ray imaging of patient comprising the steps of:
a. Positioning a patient on a turntable that is positioned between an x-ray source and an x-ray detector
b. Adjusting the position of the x-ray source and x-ray detector so that they are aligned with some portion of the region of interest
c. Rotating the turntable 360 degrees or less in continuously or fixed increments
d. Scanning the x-ray source and detector at each angular position of the turntable such that the x-ray beam intersects cumulatively the entire region of interest
e. Combining the data from all positions of the x-ray course and x-ray detector at a given angular position of the turntable
f. Combining data from all angular positions of the turntable
Whereby a 2-D image of the region of interest can be generated for each angular position of the patient and s 3-D image for all combined angular positions of the patient.

9. The method of claim 9 whereby said scanning is accomplished by adjusting the vertical and horizontal positions of said source and said detector.

10. The method of claim 9 whereby said scanning is accomplished by adjusting the vertical and horizontal positions of said source and angulating said detector.

11. The method of claim 9 whereby said scanning is accomplished by adjusting the vertical and horizontal positions of said detector and angulating said source.

12. The method of claim 9 whereby said scanning is accomplished by using a large-area scanning x-ray source whose beams converge on a small area-detector thereby allowing three dimensional data to be generated of the entire region of interest from a single angular position of the turntable.

13. The method of claim 9 whereby the imaging data can be used to monitor the position of a patient undergoing a radiation therapy treatment.

14. The method of claim 9 whereby the imaging data can be used to monitor the position of a region inside a patient undergoing a radiation therapy treatment.

15. The method of claim 9 whereby said images are two-dimensional plain x-ray images of the patient.

16. The method of claim 9 whereby said images are a CT of the patient.

17. The method of claim 9 whereby said images are three-dimensional images of the patient generated from a singular angular position of the turntable.
